(19) United States
(12) Patent Application Publication
(10) Pub. No.: US 2025/0120920 A1
(43) Pub. Date: Apr. 17, 2025

(54) DRUG-DELIVERING NERVE WRAP

(71) Applicant: UNIVERSITY OF UTAH RESEARCH FOUNDATION, Salt Lake City, UT (US)

(72) Inventors: Brett H. DAVIS, Salt Lake City, UT (US); Jill SHEA, Milcreek, UT (US); Himanshu SANT, Salt Lake City, UT (US); Pratima LABROO, Salt Lake City, UT (US); Bruce K. GALE, Taylorsville, UT (US); Jayant P. AGARWAL, Salt Lake City, UT (US)

(21) Appl. No.: 18/991,226

(22) Filed: Dec. 20, 2024

Related U.S. Application Data
(63) Continuation-in-part of application No. 17/419,227, filed on Jun. 28, 2021.
(60) Provisional application No. 62/792,727, filed on Jan. 15, 2019.

(51) Int. Cl. A61K 9/70; A61K 31/436; A61K 38/18; A61K 47/34; A61P 25/02

(52) U.S. Cl. CPC A61K 9/7007; A61K 31/436; A61K 38/185; A61K 47/34; A61P 25/02

(57) ABSTRACT

Described herein are medical film materials that incorporate one or more neuro-regenerative drugs into a polymer film. The polymer film includes a copolymer of lactide and caprolactone. The neuro-regenerative drug includes the macrolactam immunosuppressant FK506. The film is configured such that when placed under physiological conditions, the neuro-regenerative drug is released in an extended, substantially linear fashion for a period of at least 30 days.

### CROSS-REFERENCE TO RELATED APPLICATIONS

This application is a continuation-in-part of U.S. patent application Ser. No. 17/419,227, filed Jun. 28, 2021, which is a § 371 national stage entry of International Patent Application No. PCT/US2020/013498, filed Jan. 14, 2020, which claims priority to and the benefit of U.S. Provisional Patent Application No. 62/792,727, filed Jan. 15, 2019. Each of the foregoing applications is incorporated herein by reference.

### BACKGROUND

Peripheral nerve injuries can lead to loss of motor and sensory function and debilitating chronic pain, unless successful regeneration can be accomplished. The cost of peripheral nerve injuries on the American health-care system is $150 billion per year, and there are approximately 900k nerve injury procedures performed annually in the US (Taylor et al., The incidence of peripheral nerve injury in extremity trauma. Am J Phys Med Rehabil. 2008; 87(5):381-5). Only 52% of median and ulnar nerve repairs achieve satisfactory motor recovery and only 43% achieve satisfactory sensory recovery (Ruijs et al. Median and ulnar nerve injuries: a meta-analysis of predictors of motor and sensory recovery after modern microsurgical nerve repair. Plast Reconstr Surg. 2005; 116(2):484-94; discussion 95-6).

Clinically, the current gold standard for a nerve transection injury that does not result in a significant gap is to directly repair the severed nerve ends with fascicular alignment. With direct repair, currently less than 50% of patients recover meaningful function (Rujis et al.). Occasionally, nerve wraps made from polyesters or collagen are used in conjunction with direct nerve repair to prevent adhesion formation and to reduce the risk of neuroma formation. However, patient outcomes remain less than ideal and current clinically available nerve wraps have several limitations.

### BRIEF DESCRIPTION OF THE DRAWINGS

A more particular description will be rendered by the embodiments illustrated in the appended drawings. It is appreciated that these drawings depict only exemplary embodiments of the disclosure and are therefore not to be considered limiting of its scope. In the accompanying drawings:

FIGS. 1A and 1B illustrate an exemplary medical film having multiple layers, with an inner layer that incorporates one or more drugs and an outer film that omits the one or more drugs.

FIG. 1C illustrates an embodiment of a medical film loaded with one or more drugs at a concentration gradient that increases from a proximal end to a distal end.

FIG. 1D illustrates an embodiment of a medical film having surface microstructure of ridges and grooves arranged to extend in a direction of nerve growth.

FIG. 1E illustrates an embodiment of a medical film that includes pores optimized in size and shape to allow diffusion of nutrients and oxygen to underlying nerve tissue while maintaining effective structural integrity of the film.

FIG. 2 is a graph showing cumulative release profile obtained from in vitro FK506 release testing of FK506 containing PLC nerve wraps, with wraps categorized as: 0% no-drug wraps (ND-Wrap), 10% low-drug wraps (LD-Wrap), and 50% high-drug wraps (HD-Wrap). Both LD-Wrap and HD-Wraps exhibited a linear release for the first 31 days. Linear regression analysis yielded R² values for both the LD-Wrap and HD-Wrap to be R²=0.991. (n=8 for each group).

FIG. 3 is a chart showing average DRG neurite extension measured for FK506 bioactivity verification testing after fabrication and release. 0 ng/ml FK506 is the negative control group and 20 ng/ml FK506 is the positive control group. Samples from Day 4 of the drug release test were used to culture whole chick DRGs. 20 ng/ml control, Day 4 LD-Wrap, and Day 4 HD-Wrap groups were found to be significantly greater than the 0 ng/ml control group. (*p<0.05 vs 0 ng/ml).

FIG. 4 is a chart showing relative gastrocnemius muscle mass measured to assess functional recovery. The LD-Wrap group was found to have significantly greater muscle mass recovery compared to all other groups. (*p<0.05 LD-wrap vs DSR Only and HD-Wrap, p<0.01 LD-Wrap vs ND-Wrap).

FIGS. 5A-5C shows: total number of myelinated axons (FIG. 5A), nerve cross sectional area (FIG. 5B), and axon density (FIG. 5C) were determined distal to the nerve coaptation repair site. LD-Wrap was found to be significantly greater than the DSR Only ND-Wrap groups (*p<0.01). HD Wrap was found to be significantly greater than the DSR Only and ND-Wrap groups (*p<0.01). No statistically significant differences were found between the groups for the nerve fascicular area and axon density metrics.

FIG. 6 is a chart showing results of an electrophysiological assessment to assess functional recovery of the hind paw muscles. The LD-Wrap group had significantly greater relative Foot-EMG than all other groups. (*p<0.05 vs all other groups).

### DETAILED DESCRIPTION

### Introduction

Described herein are medical materials that effectively combine localized drug delivery with the functionality of an implantable medical film. In particular, described herein are nerve wraps configured for localized delivery of one or more neuro-regenerative drugs to a nerve injury site. Embodiments described herein may be utilized to treat nerve injuries, and in particular peripheral nerve injuries, to improve functional nerve regeneration outcomes while limiting or avoiding harmful side-effects associated with systemic usage of neuro-regenerative drugs.

In one embodiment, FK506 (also known as tacrolimus) is embedded in a poly (L lactic acid-co-caprolactone) polymer (“PLLA-PCL”) to create a drug-loaded film with mechanical properties that enable the film to be wrapped around nerves at a targeted nerve injury site. The film can effectively act as a barrier to surrounding tissue while simultaneously providing extended, localized delivery of FK506. Such embodiments have shown ability to provide substantially linear, near zero-order drug release kinetics in a physiological environment for time periods of at least 30 days and likely substantially longer (e.g., potentially up to about 45 days or even up to about 60 days). Films can additionally or alternatively include neurotrophic agents such as nerve growth factor (NGF) and/or brain-derived neurotrophic factor (BDNF).

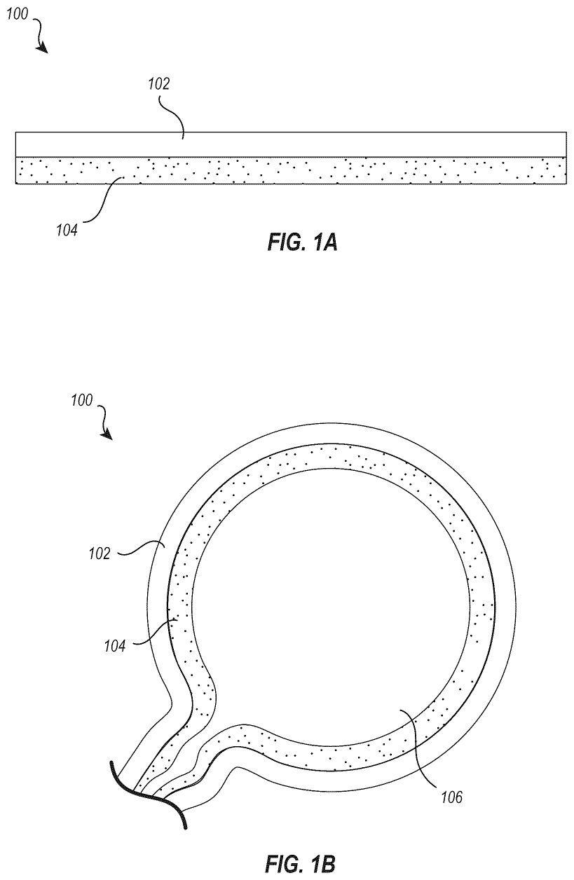

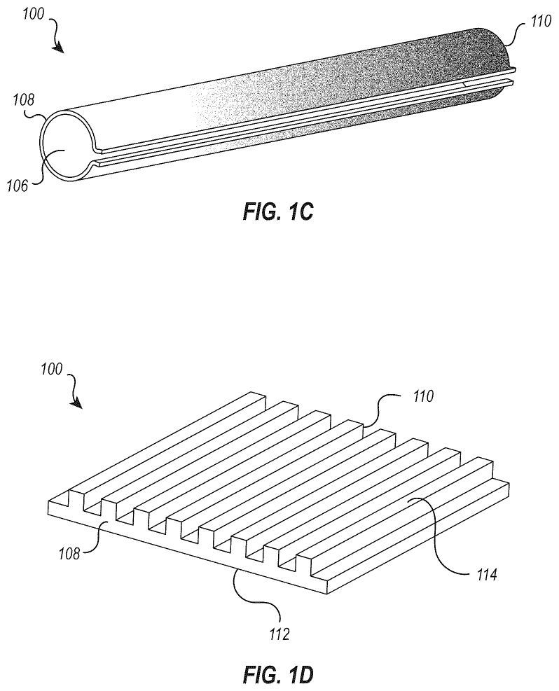

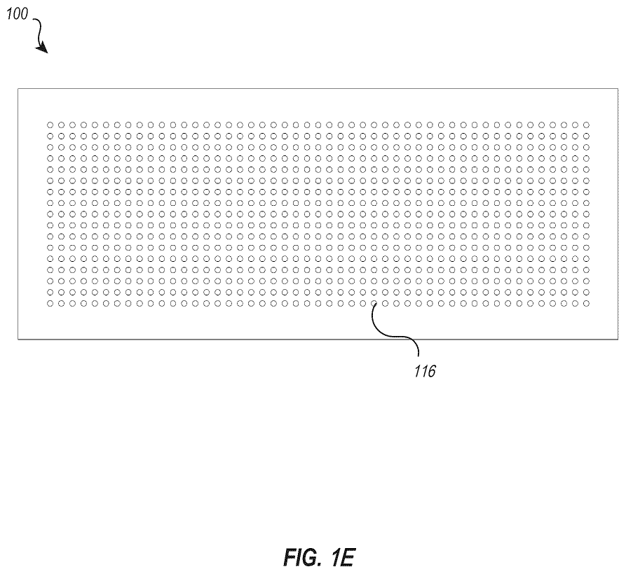

The medical films described herein may also be sometimes referred to as “wraps” since this terminology is common in applications involving a nerve injury site, though embodiments are not necessarily confined to nerve injury applications. The terms “film” and “wrap” are therefore used synonymously and are not intended to signify any structural difference in the polymer materials described. The disclosed films can be positioned to form conduits, such as shown in FIGS. 1B and 1C. Although the embodiments illustrated in these Figures are form conduits by wrapping the films into the conduit shape, it is implicit that a conduit shape can be pre-formed. The skilled person will thus understand that pre-formed conduit shapes are within the present disclosure. Accordingly, reference to films throughout this disclosure will also be understood to include conduits, whether manufactured as such or manually formed by a user (e.g., medical practitioner).

As used herein, the term “physiological environment” describes the conditions a film is exposed to when implanted into a typical subject, such as when placed at a nerve injury site. For example, physiological pH is typically about 6 to 8 (more typically neutral or slightly basic), physiological temperatures are typically about 36° to 38° C., and fluids typically have a tonicity that is isotonic (e.g., equivalent to about 0.9% w/v saline solution).

### Example Anti-Inflammatory & Neuro-Regenerative Drugs

FK506 is an FDA approved immunosuppressant and anti-inflammatory drug used to prevent allograft organ rejection. FK506 is an appealing drug candidate for use in nerve regeneration applications because it has been shown to improve functional outcomes in vivo after peripheral nerve injury via its neurotrophic effects and through reduction of scar formation. However, long-term systemic delivery of FK506 is accompanied with severe side-effects, including increased risk of infection, kidney toxicity, and liver toxicity. Localized delivery of FK506 at the site of nerve repair, such as by using a medical film embodiment described herein, has the potential to improve outcomes without the harmful side-effects associated with systemic drug use.

FK506 is relatively hydrophobic/lipophilic. As such, FK506 integrates well with relatively hydrophobic polymers. For example, FK506 has high solubility when dissolved into a polymer solution where the polymer is selected to be relatively hydrophobic. As described in more detail below, when FK506 is integrated with a relatively hydrophobic polymer to form a drug-loaded film, the substantial match in hydrophobicity provides for drug release that is highly dependent on passive diffusion out of the polymer matrix as opposed to flushing out as a bolus. This thereby enables substantially linear, zero-order release kinetics for sustained and consistent drug delivery at the nerve injury site.

Other drugs may be utilized in the film materials described herein in addition to or as an alternative to FK506. For example, some film materials may include neurotrophic agents such as NGF and/or BDNF, or can additionally or alternatively include one or more other relatively highly hydrophobic/lipophilic immunosuppressant and/or anti-inflammatory drugs such as other macrolactams or macrolactam derivatives (e.g., rapamycin, pimecrolimus, cyclosporine, ascomycin, FK506 analogs), corticosteroids, and/or non-steroidal anti-inflammatory drugs.

Preferably, a drug integrated with the film has sufficient hydrophobicity/lipophilicity to provide the above-described linear release profile when combined with the polymer to form a film material. For example, a drug integrated with the film may have one or more of: a log P (e.g., log Kow) greater than about 1.5, more preferably within a range of about 2.0 to about 5.0, or about 2.5 to 4.5, or about 3.0 to 4.2; or a water solubility (at 25° C.) of less than about 10 mg/L, less than about 5 mg/L, less than about 1 mg/L, less than about 0.1 mg/L, or less than about 0.05 mg/L.

### Example Polymer Films

The film material may be formed from a bioresorbable polymer. However, certain common bioresorbable polymers have been found to be less effective in neuro-regeneration applications. For example, the inventors found that where polylactic acid (PLA) is utilized as the polymer film, neuro-regeneration outcomes are hindered relative to other polymers tested. PLA, as used herein, refers to either of the optical isomers poly-L-lactic acid (PLLA) or poly-D-lactic acid (PDLA), or mixtures thereof. PLA is also to be distinguished from co-polymers such as PLLA-PCL that include PLA. It is thought that the degradation products of PLA inhibit nerve regeneration at the nerve injury site. Accordingly, preferred embodiments are not formed as PLA films. Poly (lactic-co-glycolic acid) (PLGA) is also less preferred. As discussed in more detail below and as demonstrated by the included working examples, films formed from PLLA-PCL and/or PCL, without other polymers such as PLA and PLGA, were found to demonstrate effective drug loading and effective drug release profiles for preferred drug agents. Many prior approaches have focused on PLA or PLGA-based polymers, without recognizing that PLLA-PCL and/or PCL polymers are capable of more effective drug release profiles, particularly with respect to the preferred drug agents (e.g., tacrolimus/FK506, NGF, and/or BDNF) and preferred loading rates (e.g., 0.01% (w/v) to 0.05% (w/v)) disclosed herein.

The polymer used to form the film preferably has an inherent viscosity (chloroform solvent, 25° C., c=0.1 g/dl) of about 0.75 to 2.0 dl/g, or about 1.0 to about 1.75 dl/g, such as about 1.5 dl/g.

In one embodiment, the polymer film is formed from a copolymer of lactide (synonymous to lactic acid for purposes of this disclosure) and caprolactone. Such copolymers (typically abbreviated as PLLA-PCL) have shown mechanical properties that make for effective use as medical films such as nerve wraps. For example, such polymers do not substantially swell when placed in a physiological environment such as a nerve injury site. As described above, this ability also allows for effective drug elution kinetics because lipophilic drugs will release based primarily on passive diffusion rather than being “flushed” out via water uptake into the polymer. Copolymers of lactide and caprolactone may also be formulated to provide effective flexibility and mechanical strength, making the films resistant to tearing or piercing. The polymer film can additionally or alternatively include PCL, which has similar advantages to PLLA-PCL. The polymer film can, for example, include PLLA-PCL, PCL, or a combination thereof.

The lactide portion of the lactide and caprolactone copolymer may be L-lactide, D-lactide, or DL-lactide, though L-lactide is preferred. For embodiments that include PLLA-PCL, the comonomer ratio (lactide to caprolactone on a molar percentage basis) may range from about 10:90 to about 90:10, or may range from about 30:70 to about 85:15, or more preferably may range from about 50:50 to about 80:20, or even more preferably may range from about 60:40 to about 75:25, such as about 70:30.

Copolymers falling within the foregoing ranges have been shown to have effective mechanical properties for nerve wrap applications. For example, nerve wraps are preferably flexible enough to be readily wrapped around nerves at a treatment site, which often requires relatively tight wrapping, while also maintaining good mechanical strength so as to avoid tearing or breaking during placement of the wrap and during the post-placement treatment period. These mechanical properties are preferably maintained even though the film may be relatively thin in construction. For example, a film thickness suitable for a nerve wrap application may be within a range of about 100 μm to about 600 μm, or about 150 μm to about 500 μm, or about 200 μm to about 400 μm.

Lactide and caprolactone copolymers with properties within the foregoing ranges are advantageously capable of forming such relatively thin films while maintaining good mechanical properties effective for nerve wrap applications. In addition, the lactide and caprolactone copolymers are advantageously capable of being loaded with hydrophobic/lipophilic drugs such as FK506 in a manner that allows for substantially linear drug release kinetics.

In some embodiments, the polymer film may include multiple layers. For example, as shown in FIGS. 1A and 1B, a film 100 may include an “outer” layer 102 and a “inner” layer 104. The inner layer 104 is loaded with the one or more neuro-regenerative drugs. The outer layer 102 is a thin layer that does not incorporate the one or more drugs. The use of multiple layers provides unidirectional drug release. For example, when the film 100 is wrapped/rolled as shown in FIG. 1B, it may be oriented so that the inner layer 104 containing the one or more drugs faces inward toward the lumen 106. In this manner, the one or more drugs will release inward into the lumen 106 while outward release will be minimized or avoided. The outer layer 102 may be applied on top of the inner layer 104 by way of heat annealing, solvent annealing, and/or other suitable manufacturing process known in the art. Alternatively, the polymer film can be single layered. The single-layered polymer film can be the only polymer layer of the nerve wrap, or at least, the only polymer layer to incorporate one or more neuro-regenerative drugs.

Additionally, or alternatively, the film 100 may be loaded with one or more drugs in a manner that provides a concentration gradient along an axial length of the film 100. For example, as shown in FIG. 1C, the one or more drugs may be loaded such that when the film 100 is in a wrapped/rolled configuration, a concentration gradient exists between a proximal end 108 and a distal end 110 of the wrap. By increasing the concentration of the one or more embedded drugs toward the distal end 110, the wrap 100 can encourage continued extension and growth of a nerve end in the distal direction.

In some embodiments, the polymer film may include a surface micropattern such as a micropattern of ridges/grooves. The inclusion of a micropattern has been shown to beneficially aid with neurite orientation and extension. For example, where a nerve wrap is used to bridge a nerve gap, axons will need to extend and bridge the gap. The use of surface micropatterns can promote neural cell orientation and guide growth of the cells along the ridges/grooves. A micropattern may be applied to a film using photolithography and/or micro-molding, for example.

An exemplary micropattern is schematically illustrated in FIG. 1D. As shown, a series of ridges and grooves may be arranged to extend along an axial direction from the proximal end 108 to the distal end 110. The ridges and grooves are positioned so that when the film 100 is wrapped/rolled, the ridges and grooves extend substantially axially (i.e., in the same direction as intended nerve growth). The ridges and grooves may be formed on a single side of the film 100. For example, the micropattern may be formed on an inner side 114 of the film 100, while an outer side 112 may omit any micropattern. When the film 100 is wrapped/rolled, the inner side 114 becomes the inner surface of the lumen 106.

A surface micropattern may be utilized such as described in Li et al., “Optimization of micropatterned poly (lactic-co-glycolic acid) films for enhancing dorsal root ganglion cell orientation and extension” Neural Regen Res. 2018 January; 13(1): 105-111. Li et al. does not describe the use of PLC films or the loading of films with a neuro-regenerative drug such as FK506. The drug-loaded PLC embodiments described herein can beneficially incorporate surface micropatterns to further increase neuro-regenerative capabilities. It is believed that in at least some circumstances, incorporating a surface micropattern in the medical films described herein may provide superior results as compared to an unloaded, PLG film such as described in Li et al.

Where a surface micropattern is utilized, the ridge and/or groove width may be within a range of about 1 μm to about 100 μm, or more preferably about 1 μm to about 30 μm, such as about 2 μm to about 20 μm or about 3 μm to about 10 μm. The width ratio of ridges to grooves may range from about 10:1 to about 1:10, but more preferably is about 5:1 to 1:5, about 2:1 to 1:2, or about 1:1.

In some embodiments, such as shown in FIG. 1E, the polymer film 100 can incorporate pores 116 extending through the thickness of the film 100. The pores 116 can beneficially enable diffusion of nutrients and oxygen to regenerating nerves underlying the film 100 (or within a wrap or conduit formed by the film 100), which can promote desired vascular and cellular integration. However, the inclusion of pores 116 can also weaken the structural integrity of the film 100, which could lead to detrimental device failure and/or disruption to intended drug release kinetics. For example, pores 116 that are too large and/or too densely positioned on the film could overly weaken the device, potentially causing the structure to degrade too quickly and/or the loaded drugs to release too abruptly. On the other hand, pores 116 that are too small and/or overly spread apart may not provide desired levels of nutrient and oxygen diffusion to the regenerating nerves.

The inventors have found that films with thickness, polymer composition, and drug loading rates as disclosed herein, and further including an average pore diameter of 10 μm to 200 μm, or 10 μm to 50 μm, or a pore diameter within a range that uses any two of the foregoing values as endpoints, with an average spacing between pores of 50 μm to 200 μm, effectively balance nutrient/oxygen exchange with structural integrity of the films. Pores smaller than this range and/or spaced farther apart than this range provided sub-optimal diffusion of nutrients and oxygen. Pores larger than this range and/or spaced closer than this range began to detrimentally affect the structure of the film 100. In contrast, films with pores 116 sized and spaced according to these ranges, when used within the physiological environment, provided desired levels of diffusion of nutrients/oxygen, to better promote vascular and cellular integration in the nerve tissue, while maintaining the structural integrity of the films for the desired treatment duration.

The polymer film 100 can incorporate and/or be coated with an anti-adherent agent. The anti-adherent agent can facilitate handling of the polymer film during manufacturing and can beneficially minimize adhesion to non-target tissues, for example. The anti-adhering agent can include, for example, stearic acid, stearate salts (e.g., magnesium, calcium), talc, silicon dioxide, glyceryl behenate, polyethylene glycols (PEGs), or combinations thereof.

### Incorporation of Drugs Into the Polymer Film

In preferred embodiments, the one or more drugs to be incorporated into the polymer film, and the polymer utilized to form the film, each have a hydrophobicity/lipophilicity that makes the drug(s) readily soluble in the polymer. In one embodiment, the one or more drugs are dissolved in a suitable organic solvent that is then added to a polymer solution prior to curing. The polymer solution containing the dissolved drug(s) may then be solvent cast into a desired film thickness. Other polymer manufacturing methods, such as melt extrusion and/or other methods known in the art, may be utilized to form the films. Curing may be carried out under vacuum and/or using other suitable curing procedures. Following curing, the films may be cut to desired sizes if not already cast to size. The films may therefore be sized to fit any size nerve or gap according to particular application needs.

A manufacturing process that includes application of heat, such as a hot-melt extrusion process, is preferably controlled to ensure that temperatures do not exceed 140° C., or in some cases stay somewhat below 140° C., to minimize or avoid thermal degradation of the incorporated drugs. The inventors discovered that FK506, in particular, is subject to thermal degradation beginning at about 140° C. when incorporated into the preferred polymers within the preferred loading ranges as disclosed herein. While conventional PLLA and PLLA co-polymer hot-melt extrusion protocols call for temperatures exceeding 140° C., limiting the maximum temperature still allows for sufficient manufacturability while minimizing or preventing unwanted thermal degradation of the FK506.

Other incorporation procedures known in the art may additionally or alternatively be utilized to incorporate the one or more drugs into the polymer. For example, at any suitable step during manufacture of the film, the one or more drugs may be contacted with the polymer by mixing, spraying, immersion, etcetera. In some embodiments, the drug(s) may be included in a monomer blend prior to and/or during polymerization of the monomers in order to incorporate the drug(s) into the resulting polymer.

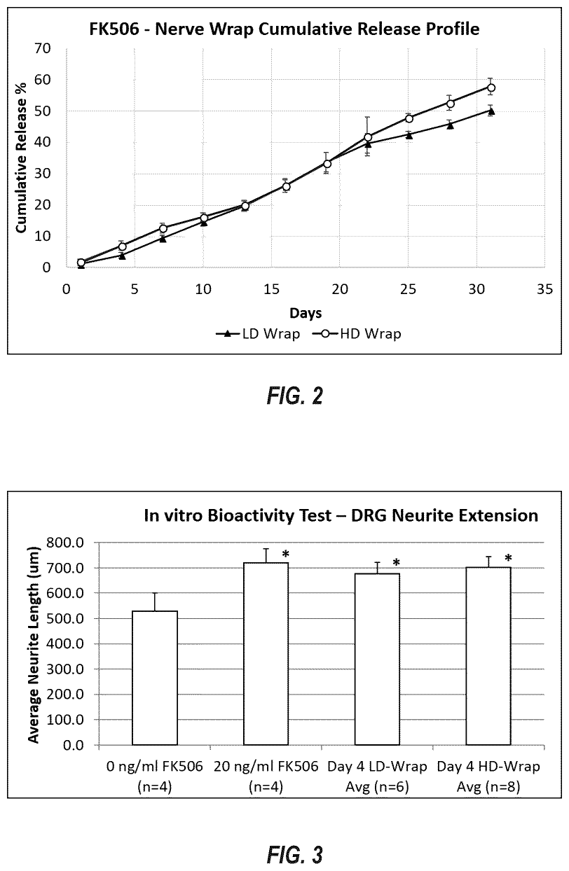

The one or more drugs may be loaded to a concentration (w/v) of about 0.001% to about 1%, or about 0.01% to about 0.1%, including about 0.05%. The foregoing ranges inherently include, for example, subranges of 0.01% to 1%, 0.05% to 0.5%, and ranges with combinations of the foregoing as endpoints, such as 0.01% to 0.05%. In some embodiments, the preferred amount of drug is less than 0.05% (w/v), such as 0.01% (w/v) to 0.04% (w/v), 0.03% (w/v), or 0.02% (w/v), because the lower levels can provide substantially similar efficacy without added cost of additional drug. See, for example, FIG. 2 and the corresponding description in the working examples. The concentration of the one or more drugs may depend on the type(s) of drugs utilized. For example, the foregoing concentration ranges may be suitable for FK506, NGF, and BDNF. However, other drugs described herein may be included at higher concentrations, such as about 2% to about 50%, or more preferably about 4% to about 30%, or about 6% to about 20%, or about 8% to about 15%. When the one or more drugs are incorporated into the polymer at concentrations within the foregoing ranges, the resulting film is able to provide effective neuro-regenerative capabilities and the beneficial elution profiles described herein.

### Drug Elution

As described above, when a neuro-regenerative drug having the characteristics described above is incorporated into a polymer having the characteristics described above, the resulting polymer film is capable of providing effective and sustained drug-release in a physiological environment such as a nerve injury site.

In at least some applications, the drug-loaded film is capable of providing substantially linear release (i.e., substantially zero-order kinetics) of the drug(s) when placed in a physiological environment for a period of at least about 10 days, or at least about 20 days, or at least about 30 days, or at least about 40 days, or at least about 50 days, or even up to at least about 60 days. A release profile may be considered “substantially linear” where a linear regression over the respective time period provides an R² value of at least 0.8, or at least 0.85, or at least 0.9, or at least 0.95, or at least 0.99.

A substantially linear drug release profile such as provided by one or more embodiments of the present disclosure provides several benefits. For example, it avoids the release of a large bolus of drug and thus limits or avoids systemic distribution of the drug. An extended, substantially linear drug release profile may also be beneficial in relatively severe nerve injury scenarios such as large compression injuries and/or those located relatively far upstream from distal end targets (e.g., upper limb injuries). In such situations, an extended, substantially linear drug release profile may particularly benefit nerve regeneration outcomes by continually promoting regeneration over longer periods of time as is often required for these injury types.

In addition, the anti-inflammatory effects of the one or more locally released drugs (such as FK506) may beneficially reduce local scar formation. This is particularly beneficial for reducing neuroma formation. This is also beneficial in the cases of nerve decompression surgery or revision nerve decompression surgery, for example, to prevent scar formation at the site of decompression.

Drug release that is too rapid can cause undesirable systemic distribution of the drug. On the other hand, drug release that is too slow may not provide desired therapeutic effects. An effective balance can be achieved where the release rate is within 0.1 ng/day to 100 ng/day, or within 1 ng/day to 20 ng/day, or is within a range with endpoints selected from any two of the foregoing values, and where that rate is maintained over a period of at least 30 days. Similarly, it is desirable to tune the release rate and total loading of the drug so that at least 50% of the drug is released within the first 30 days following implantation. This ensures that overall drug residence times are not excessive.

Films incorporating FK506 at 0.01% (w/v) to 0.05% (w/v), when positioned in the physiological environment, can beneficially provide sustained release of the FK506 over at least 30 days, with at least 50% of the drug released by 30 days. See, for example, the drug release profiles illustrated in FIG. 2 and discussed in the corresponding working examples. The combination of polymer type and drug loading beneficially function to provide effective release profiles such as demonstrated. Similar release profiles can also be achieved for other drugs such as NGF and/or BDNF.

### Methods of Use

Medical film embodiments described herein are particularly beneficial in nerve wrap applications for treating nerve injuries. Nerve wraps may be utilized, for example, in treating transected nerves (gap injuries), crushed nerves, and/or chronic nerve injuries. In some embodiments, such as in treating a gap injury, a nerve wrap may be utilized in conjunction with a direct suture repair (i.e., direct end to end repair) procedure. For example, a nerve may be repaired using epineural sutures followed by wrapping with a nerve wrap.

The nerve wraps described herein may also be utilized in conjunction with an autograft or allograft. For example, an autograft or allograft may be used to bridge a gap in a nerve, and a nerve wrap may be positioned around the autograft or allograft (and preferably also extended over the injured nerve ends). Where a nerve allograft is utilized, an immunosuppressant drug such as FK506 beneficially inhibits an immune response and thus reduces immune cell infiltration as compared to when the wrap omits the drug.

Medical films described herein may also be utilized in other applications where tissue compartmentalization and/or extended drug-release are called for. For example, a medical film as described herein may be utilized following abdominopelvic surgery to act as an anti-adherence barrier and prevent the formation of intra-abdominal adhesions. In another example, a medical film as described herein may be utilized to prevent organ and/or tissue rejection following allotransplantation. For example, the medical film may be positioned around the transplanted organ and/or tissue for extended local delivery of one or more drugs such as immunosuppressant FK506.

### Additional Terms & Definitions

Component percentages disclosed herein are based on total volume of the polymer material used to form the polymer film material, unless indicated otherwise. Thus, component amounts (e.g., drug loading amounts) are disclosed on a (w/v) basis, even though the final polymer film at time of use is a solid. Component amount percentages reported on a (w/v) basis are not substantially different from (w/w) basis based on total weight of the polymer film.

As used herein, a “neuro-regenerative drug” broadly encompasses any drug that promotes regeneration of nerve tissue, and includes anti-inflammatory drugs, immunosuppressant drugs, and neurotrophic drugs, for example. Certain drugs can impart multiple effects. FK506, for example, can impart both immunosuppressant and anti-inflammatory properties. The terms “drug” and “agent” can be used interchangeably.

While certain embodiments of the present disclosure have been described in detail, with reference to specific configurations, parameters, components, elements, etcetera, the descriptions are illustrative and are not to be construed as limiting the scope of the claimed invention.

For any given element of component of a described embodiment, any of the possible alternatives listed for that element or component may be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent (e.g., “film”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, embodiments can be essentially free or completely free of drugs and/or polymer materials not specifically disclosed herein.

An embodiment that “essentially omits” or is “essentially free of”' a component may include trace amounts and/or non-functional amounts of the component. For example, an “essentially omitted” component may be included in an amount no more than 1%, no more than 0.1%, or no more than 0.01% by total weight of the composition.

A composition that “completely omits” or is “completely free of” a component does not include a detectable amount of the component (i.e., does not include an amount above any inherent background signal associated with the testing instrument) when analyzed using standard compositional analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

### EXAMPLES

### Example 1—Nerve Wrap Fabrication

10% w/v polymer solution was made by dissolving PLC (Corbion, Amsterdam, Netherlands) in dichloromethane (Acros Organics, Geel, Belgium) and stirring at 60 rpm overnight. FK506 (PROGRAF, Astellas Pharma., Tokyo, Japan) was dissolved in 100% ethanol and added to the PLC solution to make three solutions with different concentrations of FK506:0%, 0.01%, and 0.05% (w/w FK506/PLC). From here on in this Examples section, the wraps will be identified as the 0% no-drug wraps (ND-Wrap), 0.01% low-drug wraps (LD-Wrap), and 0.05% high-drug wraps (HD-Wrap). Polymer films were formed by solvent-casting 13 ml of PLC/FK506 solutions into plastic petri dishes. Films were left to cure for 48 hours in a fume hood followed by an additional 48 hours in a vacuum. Films were cut using scissors to different sizes for the in vitro and in vivo testing, 1×1 cm and 5×3.5 mm, respectively.

### Example 2—Nerve Wrap Material Characterization

A micrometer (Fowler, Newton, Massachusetts, USA) was used to measure the thickness of the films after casting and cutting to size. A weight loss study was conducted to determine the degradation of the PLC films. 24 1×1 cm squares (8 ND-Wraps, 8 LD-Wraps, and 8 HD-Wraps) cut from the cast films were used for this study. The films were dried for 24 hours in a fume hood followed by 48 hours at vacuum, and then weighed before the study to get an initial weight. Individual films were placed into a 5 mL tube containing 3 ml of PBS and kept at 37° C. and 5% CO₂ for 8 weeks. PBS was replaced every 72 hours. At 8 weeks, the films were removed from PBS, dried in a vacuum oven for 48 hours and then weighed.

Prior to initiation of in vitro release test devices were visually inspected. The nerve wraps from all groups were qualitatively similar, as highly transparent films. Additionally, upon simple physical manipulation the wraps were smooth, flexible, and elastic films that were hard to pierce or tear. The nerve wrap's weight and thickness were then measured; the values are reported in Table 1. The average weight and thickness of all the wraps was 23.6±2.32 mg and 280±29.5 μm, respectively. Individual wraps were stored in PBS at 37° C. for 8 weeks; the PBS was changed every 72 hours. At 8 weeks the wraps were dried, weighed, and compared with initial weights to determine the relative change (Table 1).

TABLE 1

| | Weight (mg) | Thickness (μm) | Weight Change (%) |
|---|---|---|---|
| ND-Wrap (n = 4) | 22.9 ± 2.13 | 273 ± 14.2 | +8.26 ± 1.23 |
| LD-Wrap (n = 8) | 21.9 ± 1.54 | 267 ± 28.4 | +9.28 ± 2.6 |
| HD-Wrap (n = 8) | 25.7 ± 1.09 | 302 ± 24.8 | +5.59 ± 4.17 |
| Average (all groups) | 23.6 ± 2.32 | 280 ± 29.5 | +7.60 ± 3.58 |

### Example 3—FK506 Release Characterization

An in vitro release test was conducted to determine the release profile of FK506 from the PLC films. 1×1 cm squares of each PLC-FK506 nerve wrap group (4 ND-Wraps, 8 LD-Wraps, and 8 HD-Wraps) were placed in conical tubes containing 3 ml of cell culture media consisting of DMEM/F12+10% Fetal Bovine Serum (FBS) and 1% Pen-Strep (Gibco, Gaithersburg, MD, USA). Nerve wraps were stored at 37° C. and 5% CO₂ for 31 days. Cell media was collected and replaced with 3 ml of fresh media after the first 24 hours and then every 72 hours for the next 30 days. Enzyme-linked immunosorbent assays (ELISA) (Abnova, Taipei, Taiwan) were used to determine concentration of FK506 in the collected solutions for release profile determination.

This study was done to determine whether the wraps could deliver FK506 in a sustained manner for at least 30 days. A very linear release occurred over the first 31 days, linear regression analysis yielded R² values for both the LD-Wrap and HD-Wrap to be R²=0.991. At day 31, the percent cumulative release was found to be 50.1±1.69% and 57.7±2.64% for the LD-Wrap and HD-Wrap, respectively (FIG. 2).

### Example 4—Bioactivity Verification Assay

Fertilized chicken eggs (Merrills Poultry, ID, USA) were incubated at ˜39° C. under 100% relative humidity for 12 days. Dorsal root ganglions (DRG) were dissected from the embryos under a microscope. 24-well plates were coated with laminin (1 μg/ml), then 500 μL from each media sample was placed into 3 wells. DRGs were separated carefully from connective tissue for culturing and a single DRG was placed into each well. For comparison to known FK506 concentrations, DRGs were also grown in negative and positive control concentrations of FK506, 0 ng/ml and 20 ng/ml, respectively. Groups tested: 0 ng/ml FK506 (n=4), 20 ng/ml FK506 control (n=4), Day 4 collection of LD-Wrap (n=6), and Day 4 collection of HD-Wrap (n=8). amples were diluted in DMEM/F12+10% FBS and 1% Pen-Strep. HD-Wrap and LD-Wrap drug release test samples were diluted by a factor of 10 and 2, respectively. Drug release test samples average concentrations after dilution: Day 4 LD-Wrap—18.5 ng/ml FK506 and Day 4 HD-Wrap—23.1 ng/ml FK506. The plate was incubated for 72 hours at 37° C. and 5% CO₂ to evaluate the released drug's bioactivity. After culture, the DRG's were fixed with methanol and rinsed with DI water. Each DRG was imaged using a wide field light microscope with phase-contrast at 4× magnification. Images of DRGs were used to analyze neurite extension. Neurite extension measurements were done using a previously described method. Briefly, the area of the ganglion body (A_{DRG}) and the total area of the DRG with the growing axons (Aₜₒₜ) were measured using ImageJ (ImageJ 1.31v, National Institutes of Health, Bethesda, USA). The average neurite length (l_{avg}) was calculated by: l_{avg}=(Aₜₒₜ/π)^{1/2}−(A_{DRG}/π)^{1/2}.

In vitro DRG neurite extension verification testing was performed to verify that FK506 released from the nerve wraps maintained its bioactivity. Average neurite extension values observed for each group: 0 ng/ml FK506—529±72.2 μm, 20 ng/ml FK506—720±72.2 μm, Day 4 LD-Wrap—677±45.2 μm, and Day 4 HD-Wrap—702±42.1 μm. DRGs grown in the collected media from the drug release test had significantly (p<0.05) greater average neurite extension than the 0 ng/ml FK506 control group and were not significantly different than the positive control 20 ng/ml FK506 group (FIG. 3).

### Example 5—In Vivo Model and Surgical Procedure

The in vivo study protocols were executed as approved by the Institutional Animal Care and Use Committee of the University of Utah. Thirty-two adult mice (B6.Cg-Tg (Thyl-YFP) 16Jrs/J, Jackson Laboratory) were used for this experiment. Mice were divided into four experimental groups: ND-Wrap, LD-Wrap, and HD-Wrap and control direct suture repair with no wrap (DSR Only) group, with eight mice in each group. Mice were anesthetized with isoflurane. The surgical area on the right hind limb was shaved and prepared with alcohol and betadine. A longitudinal incision was made in the posterior distal thigh of the hind limb, separating the natural muscle planes. The sciatic nerve was isolated and transected immediately proximal to its bifurcation into the tibial and peroneal nerves. The transected ends of the nerve were then repaired using 2 9-0 nylon epineural sutures. The nerve wrap was then placed around the direct suture repair site of the experimental groups. Three sutures were then used to close the wrap around the nerve by suturing it to itself after wrapping with one at each end and one in the middle of the wrap. An extra suture was used on the distal end to fix the wrap to the nerve. Animals were sacrificed at 6 weeks for electrophysiological assessment and tissue harvest.

### Example 6—Gastrocnemius Muscle Mass Assessment

The gastrocnemius muscle of both hind legs was harvested at necropsy by careful to dissection at the tendinous origin and insertion points. The muscles were weighed and the relative muscle mass of the experimental leg was calculated by comparing the weight to the contralateral side: Relative % Gastrocnemius Muscle Mass=(Mass_{Experimental}/Mass_{Contralateral})×100.

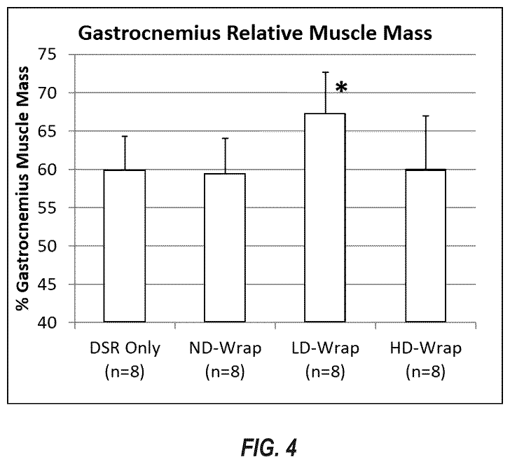

Six weeks following sciatic nerve transection and repair, the animals were sacrificed and bilateral gastrocnemius muscles from each animal were surgically removed and weighed. Relative masses between the experimental and non-injured sides were calculated: DSR Only—59.8±4.48%, ND-Wrap—59.4±4.70%, LD-Wrap—67.2±5.44%, and HD-Wrap—60.0±6.99% (FIG. 4). The LD-Wrap group had significantly greater muscle mass when compared to all other groups: DSR only (p<0.05), ND-Wrap (p<0.01), and HD-Wrap (p<0.05).

### Example 7—Paraffin Embedding and Axon Quantification

At animal sacrifice, the sciatic nerve with wrap left intact were harvested, fixed in formalin for 24 hours, and then transferred to 2% glycine for storage prior to osmium staining and paraffin embedding. At the time of embedding, the nerves were post-fixed in osmium tetroxide (2%) for 90 minutes, dehydrated, and paraffin embedded. 3 μm thick sections were obtained using a microtome and then stained with hematoxylin and eosin (H&E). A ZEISS Axio Scan.Z1 (Oberkochen, Germany) was used to image the sections. Analysis was performed using ImageJ to determine nerve fascicle area, axon density, and total number of myelinated axons. Stereological techniques were used to obtain unbiased representations of the total number of myelinated axons and axon diameter per cross section.

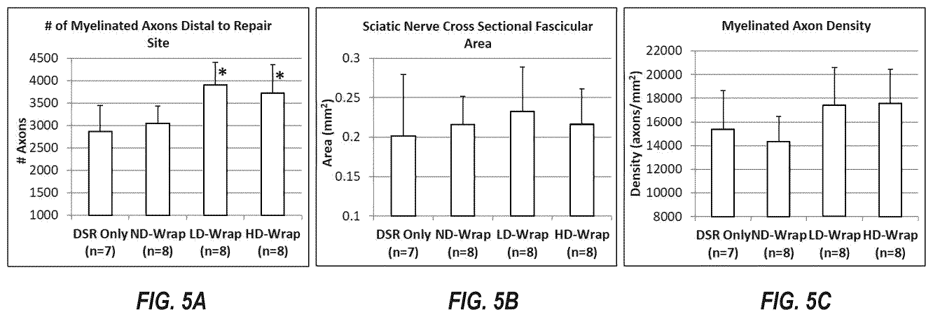

Nerve regeneration distal to the injury was assessed by comparing number of myelinated axons across groups. The average total number of myelinated axons per group are as follows: DSR Only=2870±578 axons, ND-Wrap=3050±382 axons, LD-Wrap=3910±502 axons, and HD-Wrap=3720±635 axons (FIG. 5A). Both drug containing wrap groups (LD-Wrap and HD-Wrap) had a significantly (p<0.01) greater number of myelinated axons than both the DSR only group and ND-Wrap group. The average sciatic nerve fascicular area is as follows: DSR Only=0.201±0.0782 mm², ND-Wrap=0.216±0.0358 mm², LD-Wrap=0.233±0.0563 mm², and HD-Wrap=0.216±0.0444 mm²(FIG. 5B). The average axon density is as follows: DSR Only=15,400±3290 axons/mm², ND-Wrap=14,300±2150 axons/mm², LD-Wrap=17,400±3170 axons/mm², and HD-Wrap=17,600±2900 axons/mm² (FIG. 5C).

### Example 8—Electrophysiological Assessment

Electrophysiological assessment was performed immediately prior to sacrificing of the animals to assess the functional recovery of the motor end-targets. Animals were anesthetized with isoflurane and shaved. The right sciatic nerve was exposed similar to the implantation procedure, and the site of injury/repair was located. A custom fabricated pair of stimulating hook electrodes was placed proximal to the repair site. The hind limb was coated with conductive gel, and a stainless-steel ring surface electrode (Natus Neurology, Middleton, WI, USA) was placed over Achilles tendon. Additionally, a cup electrode (Natus Neurology, Middleton, WI, USA) was clipped onto the center of the foot. The nerve was stimulated with a supramaximal 0.1 ms duration pulse and surface electromyograms (EMG) were recorded. The differential signal between the Achilles ring electrode and the foot cup electrode were amplified, filtered, recorded, and analyzed to determine the peak-to-peak amplitude for each signal. This process was then repeated for the left hind limb to serve as a contralateral control.

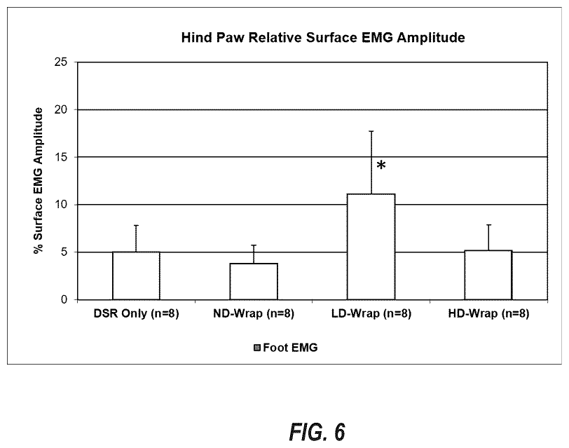

Electrophysiological assessment of the reinnervation of the plantar muscles was performed by recording surface EMG signals from the hind paw region (Foot-EMG). Average Foot-EMG values normalized to the contralateral leg: DSR Only—4.99±2.84%, ND-Wrap—3.84±1.89%, LD-Wrap—11.1±6.65% axons, and HD-Wrap—5.17±2.69% (FIG. 6). The LD-Wrap group had a significantly (p<0.05) greater Foot-EMG response than all other groups.

### Statistical Analysis

The data from the in vitro drug release test was analyzed with a linear regression trendline analysis. The DRG neurite extension assay was analyzed with the Student's t-test. The data from the in vivo study was screened for outliers, tested for normality, and analyzed with a one-way ANOVA with a Student's t-test post-hoc analysis. Outliers were defined as being outside of Q₁/Q₃+1.5 times the interquartile range and were replaced with the mean. Data was verified using Anderson-Darling, Jarque-Bera, and Lilliefors tests for normality. No groups were found to be nonparametric. Data groups with p<0.05 were considered significant.

1. A drug-eluting nerve wrap for regeneration of targeted nerve tissue, the drug-eluting nerve wrap comprising:
a flexible polymer film configured in size and shape to surround targeted nerve tissue;
an anti-inflammatory drug incorporated into the polymer film and included at 0.01% (w/v) to 1% (w/v) based on total volume of polymer formed into the nerve wrap;
a neurotrophic agent incorporated into the polymer film and included at 0.01% (w/v) to 1% (w/v) based on total volume of polymer formed into the nerve wrap;
wherein, when placed in a physiological environment, the drug-eluting wrap is configured to provide
(i) sustained release of the anti-inflammatory drug at 0.1 ng/day to 100 ng/day over a period of at least 30 days, and
(ii) sustained release of the neurotrophic agent at 0.1 ng/day to 100 ng/day over a period of at least 30 days.

2. The nerve wrap of claim 1, wherein at least 50% of the anti-inflammatory drug and/or the neurotrophic agent is released within the first 30 days following placement of the nerve wrap within the physiological environment.

3. The nerve wrap of claim 1, wherein the nerve wrap is configured as a conduit.

4. The nerve wrap of claim 1, wherein the anti-inflammatory drug comprises FK506.

5. The nerve wrap of claim 4, wherein the FK506 is included at 0.01% (w/v) to 0.05% (w/v).

6. The nerve wrap of claim 1, wherein the neurotrophic agent comprises nerve growth factor (NGF), brain-derived neurotrophic factor (BDNF), or both.

7. The nerve wrap of claim 1, wherein the polymer film comprises poly (lactic acid-co-caprolactone) (PLLA-PCL), polycaprolactone (PCL), or combination thereof.

8. The nerve wrap of claim 8, wherein the PLLA-PCL has a lactide to caprolactone ratio, on a molar percentage basis, that ranges from 60:40 to 75:25.

9. The nerve wrap of claim 1, wherein the polymer film consists of PLLA-PCL, PCL, or combination thereof.

10. The nerve wrap of claim 1, wherein the polymer film omits PLA and PLGA.

11. The nerve wrap of claim 1, wherein the polymer film has a thickness within a range of 100 μm to 600 μm.

12. The nerve wrap of claim 1, wherein the polymer film is configured to provide substantially linear release of the anti-inflammatory drug and neurotrophic agent over a period of at least 30 days when placed in a physiological environment.

13. The nerve wrap of claim 1, wherein the polymer film includes pores distributed across the polymer film.

14. The nerve wrap of claim 14, wherein the pores include an average pore diameter of 10 μm to 200 μm, with an average spacing between pores of 50 μm to 200 μm.

15. The nerve wrap of claim 1, wherein the polymer film is single-layered and wherein the single-layered polymer film is the only polymer layer of the nerve wrap.

16. A drug-eluting nerve wrap for regeneration of targeted nerve tissue, the drug-eluting nerve wrap comprising:
a flexible polymer film configured in size and shape to surround targeted nerve tissue, wherein the polymer film comprises poly (lactic acid-co-caprolactone) (PLLA-PCL), polycaprolactone (PCL), or combination thereof, wherein the polymer film includes pores distributed across the polymer film, and wherein the pores include an average pore diameter of 10 μm to 50 μm with an average spacing between pores of 50 μm to 200 μm;
an anti-inflammatory drug comprising FK506 incorporated into the polymer film at 0.01% (w/v) to 0.05% (w/v) based on total volume of polymer that forms the polymer film;
a neurotrophic agent comprising nerve growth factor (NGF), brain-derived neurotrophic factor (BDNF), or both, incorporated into the polymer film at 0.01% (w/v) to 1% (w/v) based on total volume of polymer that forms the polymer film;
wherein, when placed in a physiological environment, the drug-eluting wrap is configured to provide
(i) sustained release of the anti-inflammatory drug at 0.1 ng/day to 100 ng/day over a period of at least 30 days, with at least 50% of the anti-inflammatory drug being released over the period of at least 30 days, and
(ii) sustained release of the neurotrophic agent at 0.1 ng/day to 100 ng/day over a period of at least 30 days, with at least 50% of the neurotrophic agent being released over the period of at least 30 days.

17. A method of treating an injured nerve, comprising:
providing the nerve wrap of claim 1; and
placing the nerve wrap at a nerve injury site such that the nerve wrap surrounds the targeted nerve tissue.

18. The method of claim 17, wherein the nerve injury site is a gap injury.

19. The method of claim 18, wherein the gap injury is repaired with a direct end to end repair.

20. The method of claim 18, wherein the gap injury is repaired using an autograft or allograft.
